(19) United States
(12) Patent Application Publication
(10) Pub. No.: US 2023/0351538 A1
(43) Pub. Date: Nov. 2, 2023

(54) FALL RISK DETERMINATION APPARATUS, FALL RISK DETERMINATION METHOD, AND PROGRAM

(71) Applicant: NIPPON TELEGRAPH AND TELEPHONE CORPORATION, Tokyo (JP)

(72) Inventors: Rie SAKAI, Musashino-shi, Tokyo (JP); Hiroshi YOSHIDA, Musashino-shi, Tokyo (JP); Tomoko SHIBATA, Musashino-shi, Tokyo (JP)

(73) Assignee: NIPPON TELEGRAPH AND TELEPHONE CORPORATION, Tokyo (JP)

(21) Appl. No.: 18/026,112

(22) PCT Filed: Sep. 18, 2020
(86) PCT No.: PCT/JP2020/035557

§ 371 Date: Mar. 13, 2023

(51) Int. Cl. G06Q 50/26; A61B 5/11; A61B 5/16; A61B 5/00

(52) U.S. Cl. CPC G06Q 50/265; A61B 5/1121; A61B 5/165; A61B 5/7275; A61B 2503/20

(57) ABSTRACT

The falling risk determination device includes the acquisition unit, the calculation unit, the measurement unit, and the determination unit. The acquisition unit acquires time-series data related to gravity center oscillation of the worker from a sensor unit provided at a leg portion of a high-place work tool on which the worker rides. The calculation unit calculates an evaluation value related to the gravity center oscillation from the time-series data. The measurement unit carries out a Stroop test on the worker in parallel with processing of acquiring time-series data, and measures the fatigue level of the worker using the result of the Stroop test. The determination unit determines that the falling risk is high in a case where the evaluation value is larger than an average value of past evaluation values corresponding to the fatigue level.

### TECHNICAL FIELD

An aspect of the present invention relates to a falling risk determination device, a falling risk determination method, and a program.

### BACKGROUND ART

Injury accidents during high-place work such as electric communication construction have become a problem, and in particular, a certain number of accidents related to falling of a worker occur every year. Therefore, there is a demand for a technique for identifying dangerous motions such as unsteadiness and falling of a worker.

For example, there is a technique in which a pressure sensor having a plurality of measurement points is arranged on an object on a plane, and a motion of a worker is identified from a characteristic point of pressure when the worker performs a motion on the object on the plane where the pressure sensor is arranged (e.g., refer to Patent Literature 1). There is also a planar sheet on which a plurality of sensors is arranged in advance (e.g., refer to Non Patent Literature 1). When the worker works on the planar sheet, it is possible to identify dangerous motions such as falling.

### CITATION LIST

### Patent Literature

Patent Literature 1: JP 2006-223651 A

### Non Patent Literature

Non Patent Literature 1: Inc. Anima, “BALANCE CODER BW-6000”, [online], [searched on Sep. 9, 2019], Internet <URL: https://anima.jp/products/bw6000/>

### SUMMARY OF INVENTION

### Technical Problem

It is not realistic from the viewpoint of safety and cost that the worker works on an object or a sheet on which a sensor is arranged, since it is different from working on a normal scaffold or rug.

The present invention has been made in view of the above circumstances, and an object of the present invention is to provide a falling risk determination device, a falling risk determination method, and a program capable of determining a falling risk of a worker in high-place work.

### Solution to Problem

A falling risk determination device according to an aspect of the present invention includes an acquisition unit, a calculation unit, a measurement unit, and a determination unit. The acquisition unit acquires time-series data related to the gravity center oscillation of the worker from a sensor unit provided at a leg portion of a high-place work tool on which the worker rides. The calculation unit calculates an evaluation value related to the gravity center oscillation from the time-series data. The measurement unit carries out a Stroop test on the worker in parallel with the processing of acquiring the time-series data, and measures the fatigue level of the worker using the result of the Stroop test. The determination unit determines that the falling risk is high in a case where the evaluation value is larger than an average value of past evaluation values corresponding to the fatigue level.

### Advantageous Effects of Invention

According to an aspect of the present invention, it is possible to provide a falling risk determination device, a falling risk determination method, and a program capable of determining a falling risk of a worker in high-place work.

### BRIEF DESCRIPTION OF DRAWINGS

FIG. 1 is a block diagram of a falling risk determination system according to a first embodiment of the present invention.

FIG. 2 is a diagram for explaining a stepladder including a sensor unit.

FIG. 3 is a diagram for explaining a relationship between work on a stepladder and a mind and body function.

FIG. 4 is a flowchart for explaining an operation of a falling risk determination device.

FIG. 5 is a diagram for explaining a falling risk determination operation by a determination unit.

FIG. 6 is a diagram for explaining an example of falling risk information.

FIG. 7 is a flowchart for explaining an operation of a falling risk determination device according to a second embodiment of the present invention.

FIG. 8 is a diagram for explaining a falling risk determination operation by a determination unit.

FIG. 9 is a diagram for explaining an example of falling risk information.

FIG. 10 is a flowchart for explaining an operation of a falling risk determination device according to a third embodiment of the present invention.

FIG. 11 is a diagram for explaining a falling risk determination operation by a determination unit.

### DESCRIPTION OF EMBODIMENTS

The following description will explain embodiments of the present invention with reference to the drawings. In the following description, elements having the same functions and configurations are denoted by the same reference numerals, and redundant description will be omitted.

### [1] First Embodiment

### [1-1] Configuration of Falling Risk Determination System 1

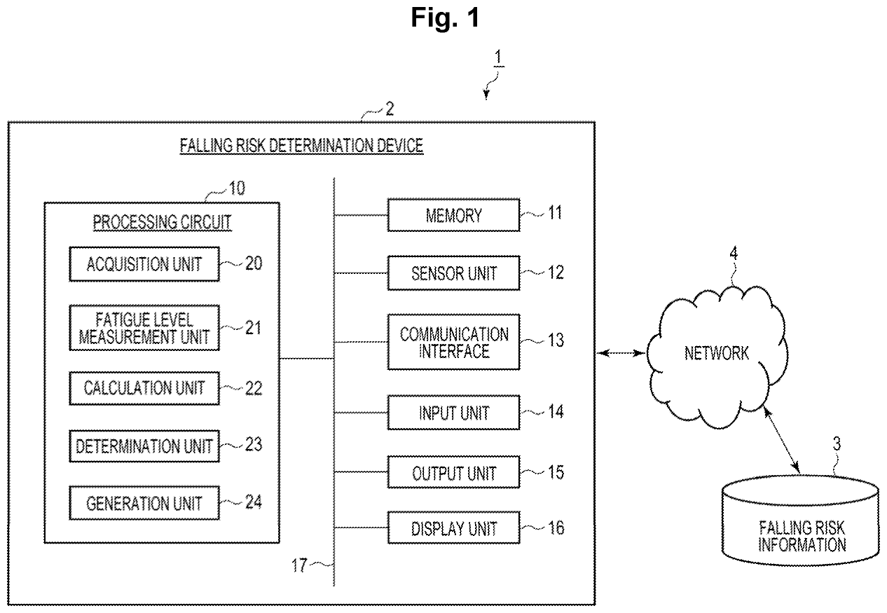

FIG. 1 is a block diagram of a falling risk determination system 1 according to a first embodiment of the present invention. The falling risk determination system 1 includes a falling risk determination device 2 and a falling risk information database 3.

The falling risk determination device 2 and the falling risk information database 3 are connected in a wired or wireless manner via a network 4. Note that, although one falling risk determination device 2 is illustrated in the example in FIG. 1, a plurality of falling risk determination devices 2 may be connected with one falling risk information database 3.

The falling risk determination device 2 includes a processing circuit 10, a memory 11, a sensor unit 12, a communication interface 13, an input unit 14, an output unit 15, and a display unit 16. The processing circuit 10, the memory 11, the sensor unit 12, the communication interface 13, the input unit 14, the output unit 15, and the display unit 16 are connected with each other via a bus 17. Note that the sensor unit 12 may be connected with the processing circuit 10 in a wired or wireless manner via the communication interface 13.

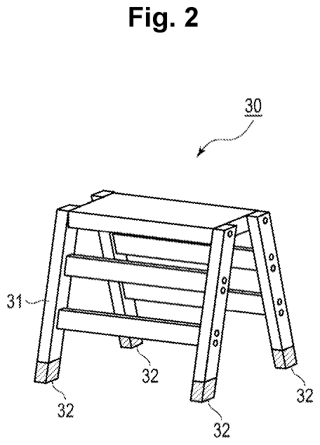

The sensor unit 12 includes a plurality of sensors, and the plurality of sensors is dispersedly arranged on a leg portion of a high-place work tool on which the worker rides so as to calculate the gravity center of the worker. Although the high-place work tool will be described on the assumption of a stepladder in the present embodiment, the high-place work tool may be any tool as long as the tool can be used when a worker rides on the tool and works at a position higher than the ground, such as a tripod, a workbench, and a scaffold stand. The sensor unit 12 acquires a sensor value that changes according to the movement of the gravity center of the worker. A sensor used as the sensor unit 12 is, for example, a strain sensor capable of measuring a pressure value. Note that an arrangement example of the sensor unit 12 will be described later with reference to FIG. 2.

The processing circuit 10 is a circuit that performs control for implementing the function of the falling risk determination device 2. The processing circuit 10 includes an acquisition unit 20, a fatigue level measurement unit 21, a calculation unit 22, a determination unit 23, and a generation unit 24.

The acquisition unit 20 acquires a worker ID from an ID recognition tag detected by the sensor unit 12. Moreover, the acquisition unit 20 acquires a sensor value related to the weight of the worker from the sensor unit 12, and acquires a time when the worker rides on the stepladder from the sensor unit 12. Furthermore, the acquisition unit 20 acquires time-series data of the sensor value.

The calculation unit 22 calculates the gravity center of the worker from the sensor value (time-series data) and calculates an evaluation value related to gravity center oscillation. The gravity center oscillation indicates fluctuation of the body gravity center in an upright posture. The evaluation value related to the gravity center oscillation is, for example, a gravity center oscillation area. The gravity center oscillation area is an outer peripheral area of the locus of the gravity center position.

The fatigue level measurement unit 21 carries out a Stroop test. In the present embodiment, the fatigue level of the worker is measured using the Stroop test. Then, the fatigue level measurement unit 21 calculates a Stroop test score using the test result.

The Stroop test is a task reported by an American psychologist Stroop and others in 1935, in which a participant is taught to answer the color of written characters. In a case where the meaning of the characters has a relationship with a color and is different from said color (mismatched characters), the participant shows difficulty. For example, it is a case where a participant is required to answer the color of red characters “BLUE”, or the color of green characters “YELLOW”. This is because the meaning of the characters hinders answering the color of the characters, and the participant must suppress the tendency to answer the meaning of the characters (dominant action).

The determination unit 23 determines the falling risk. That is, the determination unit 23 compares a measured value of the current Stroop test with an average value of past Stroop tests, and determines the falling risk according to the comparison result. The measured value is the gravity center oscillation area in the current Stroop test. The average value is an average value of gravity center oscillation areas in past Stroop tests. The average value of past gravity center oscillation areas corresponds to a usual gravity center oscillation area of the worker.

The generation unit 24 generates falling risk information. The falling risk information includes, for example, a current measured value, a determination result of the falling risk, and an average value of past gravity center oscillation areas.

Note that the processing circuit 10 includes a processor such as a central processing unit (CPU), or an integrated circuit such as an application specific integrated circuit (ASIC). Each of the above-described processing units (acquisition unit 20, fatigue level measurement unit 21, calculation unit 22, determination unit 23, and generation unit 24) may be implemented as one function of a processor or an integrated circuit by the processor or the integrated circuit executing a processing program.

The memory 11 stores data such as a sensor value, a fatigue level, an evaluation value, and worker identification information. The memory 11 may be a generally used storage medium such as a hard disk drive (HDD), a solid state drive (SSD), and a flash memory. Moreover, if the falling risk determination device 2 can transmit and receive data to and from the falling risk information database 3 via the network 4, data (sensor value, fatigue level, evaluation value, worker identification information, etc.) may be transmitted to the falling risk information database 3 every time the falling risk determination device 2 acquires and generates the data, and the memory 11 may not hold past data. In this case, the memory 11 may be a temporary storage medium configured with a volatile memory such as a cache memory.

The communication interface 13 is an interface for data communication among the sensor unit 12, the falling risk information database 3, and the falling risk determination device 2. As the communication interface 13, a generally used communication interface can be used.

The input unit 14 accepts input information from the worker. The input unit 14 includes a mouse, a keyboard, a switch, a button, a touch panel display, a microphone, and the like.

The output unit 15 outputs various types of information generated by the processing circuit 10 to the outside. For example, the output unit 15 outputs various types of information generated by the processing circuit 10 to the falling risk information database 3 via the communication interface 13. Moreover, the output unit 15 outputs a report regarding the falling risk information to the falling risk information database 3 via the communication interface 13.

The display unit 16 displays various types of information generated by the processing circuit 10. The worker can visually recognize the information by viewing the screen of the display unit 16. The display unit 16 includes a liquid crystal display (LCD) device, an organic electro-luminescence (EL) display device, or the like.

The falling risk information database 3 stores the fatigue level, the evaluation value, the worker identification information, and the like transmitted from the falling risk determination device 2. Specifically, the falling risk information database 3 stores a current fatigue level, an average value of past fatigue levels, a current evaluation value, an average value of past evaluation values, and the like. In the present embodiment, the fatigue level is a Stroop test score. The evaluation value is a gravity center oscillation area. Moreover, the falling risk information database 3 stores falling risk information to be reported to the worker. The falling risk information database 3 is assumed to be prepared in a cloud server, for example, and communicate with a plurality of falling risk determination devices 2, or may be stored in a dedicated server.

(Configuration of Sensor Unit 12)

Next, an example of a stepladder which is a high-place work tool on which the worker rides, and the sensor unit 12 attached to the stepladder will be described. FIG. 2 is a diagram for explaining a stepladder 30 including the sensor unit 12.

The sensor unit 12 includes a sensor 32 arranged on each leg 31 of the stepladder 30 on which the worker rides. For example, it is assumed that each sensor 32 is attached to a tip portion of each leg 31 of the stepladder 30. Since an anti-slip grip made of rubber or the like is usually provided at the tip portion of each leg 31, each sensor 32 may be arranged between an anti-slip grip and the tip portion of a leg 31, each sensor 32 may be embedded in the anti-slip grip itself, or a member having an anti-slip function including each sensor unit 12 may be provided at the tip portion of a leg 31 from above the anti-slip grip.

Although it is assumed that each sensor 32 acquires the pressure value as a sensor value, other information such as a sensed time, an altitude, a temperature, or a magnetic field may be acquired as the sensor value. Each sensor 32 can measure a weight, and includes, for example, a strain sensor capable of measuring a weight.

In the example in FIG. 2, four sensors 32 are each arranged on a leg 31, so that the pressure generated when the worker rides on the stepladder 30 can be acquired as a sensor value from each sensor 32. Since the pressure applied to the sensor 32 fluctuates when the worker rides on the stepladder 30, it is possible to detect that the worker rides on the stepladder 30. Furthermore, time-series data of the sensor value is obtained by continuously acquiring the sensor value from each position of the four sensors 32 at regular intervals. The fluctuation of the gravity center of the worker can be calculated using this time-series data. Note that, although four sensors 32 are attached respectively to the four legs 31 of the stepladder 30 in the sensor unit 12, the number of the sensors 32 is not limited to four, and may be any of three or more. As long as the number of the sensors 32 is three or more, it is possible to detect the fluctuation of the gravity center of the worker.

Moreover, each sensor unit 12 includes a tag recognition unit that detects an ID recognition tag held by the worker. The ID recognition tag includes information on a worker ID for uniquely identifying the worker. The sensor unit 12 recognizes the ID recognition tag of a worker who is about to get on the stepladder 30 for work, and acquires the worker ID of the worker who is on the stepladder 30. The method of recognizing the ID recognition tag by the sensor unit 12 may be, for example, a configuration of causing the worker to bring the ID recognition tag close to or into contact with the sensor unit 12, or a configuration in which the sensor unit 12 can recognize the ID recognition tag present within a certain range from the sensor unit 12. Note that the worker ID of the worker who is on the stepladder 30 may be identified by causing the worker to input his/her own ID to the input unit 14 of the falling risk determination device 2 before working, instead of identifying the worker ID by the ID recognition tag.

### [1-2] Operation

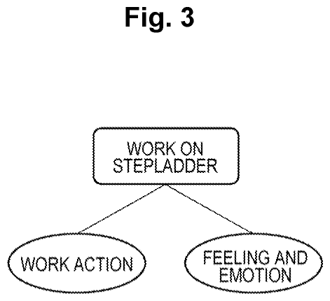

FIG. 3 is a diagram for explaining a relationship between work on a stepladder and a mind and body function. Work on a stepladder is divided into “work action” and “feeling and emotion”.

The work action includes “difficult”, “losing body balance”, and the like. The work action can be measured using the gravity center oscillation area. That is, the stability of the posture can be determined from the relative relationship between the magnitude of the stability limit and the magnitude of the body oscillation.

Feeling and emotion include “panicking”, “tired”, and the like. Feeling and emotion can be measured using the fatigue level of the worker. The fatigue level and the mental tension state cause a change in the gravity center oscillation area. Therefore, attention is paid to the gravity center oscillation and the fatigue level in the present invention. Then, the falling risk of the worker is determined using the gravity center oscillation and the fatigue level.

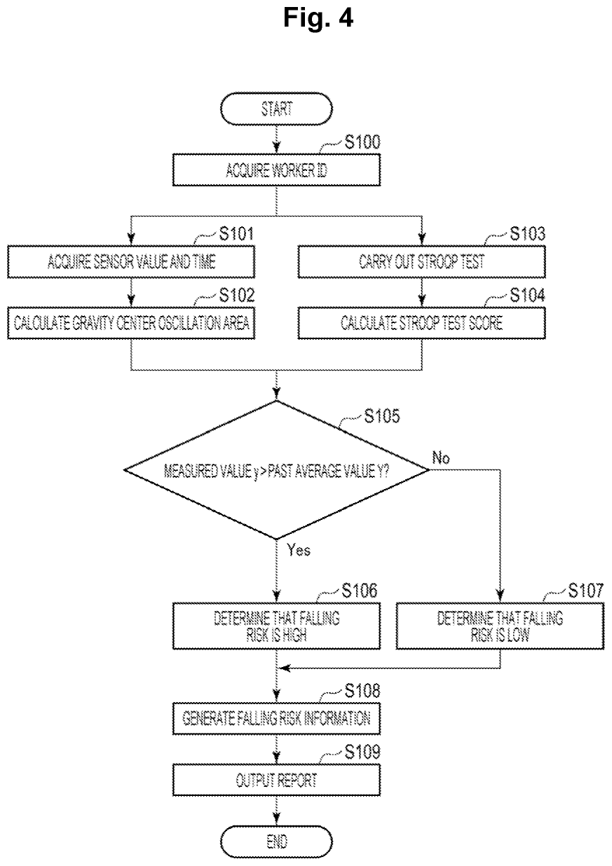

Next, the operation of the falling risk determination device 2 will be described. FIG. 4 is a flowchart for explaining an operation of the falling risk determination device 2.

The sensor unit 12 detects an ID recognition tag held by the worker. The acquisition unit 20 acquires the worker ID from the ID recognition tag detected by the sensor unit 12 (step S100). As a result, the processing circuit 10 recognizes the worker to be measured this time.

Then, the acquisition unit 20 acquires a sensor value related to the weight of the worker from the sensor unit 12, and acquires a time when the worker rides on the stepladder from the sensor unit 12 (step S101). The processing circuit 10 sets the time acquired in step S101 as a Stroop test start time. In a case where work is performed after the worker inputs his/her own ID to the input unit 14 of the falling risk determination device 2, the time when the worker ID is inputted is set as the Stroop test start time. Moreover, the acquisition unit 20 continuously acquires the sensor value at regular intervals so as to acquire time-series data of the sensor value. The sensor value changes according to movement of the gravity center of the worker.

Then, the calculation unit 22 calculates the gravity center of the worker from the sensor value (time-series data) and calculates an evaluation value related to gravity center oscillation (step S102). The evaluation value related to the gravity center oscillation is, for example, a gravity center oscillation area. When the sensor values of the respective legs of the stepladder are equal, the calculated gravity center of the worker shows that the gravity center of the worker is at the center of the planar region defined by the arrangement of the four sensors (e.g., the center of a work region of the worker defined by the four legs of the stepladder). Therefore, by comparing the fluctuation of the respective sensor values, it is possible to calculate where in the planar region the gravity center of the worker is.

Moreover, a general method, such as using the outer peripheral area of the locus of the gravity center position, may be used for calculating the gravity center oscillation area, and thus, the description thereof is omitted here. In a case where the evaluation value is the maximum value of the fluctuation width in each axis direction of the gravity center locus, the maximum value and the minimum value of the coordinates in the vertical direction and the horizontal direction may be calculated with respect to the calculated gravity center to obtain a difference, and the fluctuation width may be calculated.

In parallel with steps S101 and S102, the fatigue level measurement unit 21 carries out a Stroop test (step S103). The worker on the stepladder views the display unit 16 of the falling risk determination device 2 and carries out the Stroop test. In the Stroop test, a plurality of questions are displayed on the display unit 16 at regular time intervals. The worker views a question appearing on the display unit 16 and answers the color with voice. A microphone included in the input unit 14 acquires the voice of the worker. Note that the worker may answer by pressing a button included in the input unit 14.

The answer result of the worker is stored in the memory 11. The fatigue level measurement unit 21 compares the answer result of the worker with a correct answer stored in advance in the memory 11, and calculates the correct answer rate of the worker and the time taken for answering. The fatigue level measurement unit 21 calculates the Stroop test score using the test result (step S104). The Stroop test score corresponds to the fatigue level of the worker. The Stroop test score is a number of 11 stages from 0 to 10, where 0 indicates the lowest fatigue level and 10 indicates the highest fatigue level.

Note that a PC (test PC) for the Stroop test may be separately prepared, and the worker may carry out the Stroop test using this test PC. In this case, the fatigue level measurement unit 21 acquires the test result from the test PC.

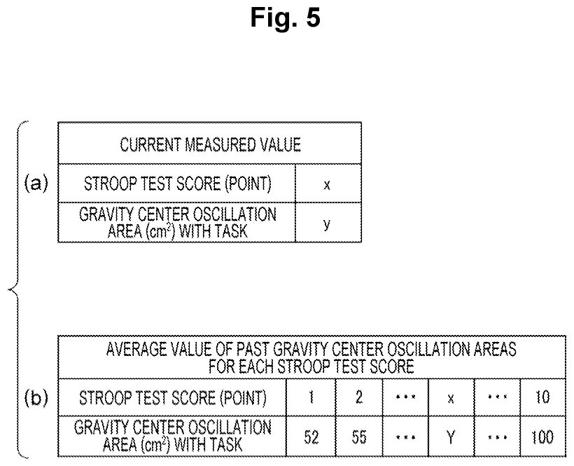

Then, the determination unit 23 determines the falling risk. FIG. 5 is a diagram for explaining a falling risk determination operation by the determination unit 23. FIG. 5(a) illustrates a current measured value, and FIG. 5(b) illustrates an average value of past gravity center oscillation areas for each Stroop test score. The current Stroop test score (point) is denoted by “x”, and the gravity center oscillation area (cm²) with a Stroop test (also referred to as task) is denoted by “y”. The average value of gravity center oscillation areas with a task is denoted by “Y”. “With a task” means that a Stroop test has been carried out. “Without a task” means that a Stroop test has not been carried out.

As illustrated in FIG. 5(b), in a case where the Stroop test score is 1 as an example, the average value of past gravity center oscillation areas is 52. In a case where the Stroop test score is 2, the average value of past gravity center oscillation areas is 55. In a case where the Stroop test score is 10, the average value of past gravity center oscillation areas is 100.

When the current Stroop test score is “x”, the determination unit 23 compares the current gravity center oscillation area (measured value) “y” with a task, with the average value “Y” of past gravity center oscillation areas with a task of when the Stroop test score is the same “x” (step S105). The average value of past gravity center oscillation areas for each Stroop test score is stored in the memory 11.

Note that the falling risk information database 3 stores an average value of past gravity center oscillation areas for each worker ID. In a case where a worker ID is recognized in step S100, the determination unit 23 stores the average value of past gravity center oscillation areas related to the worker ID from the falling risk information database 3 to the memory 11. Moreover, the memory 11 may store the same data as the average value of past gravity center oscillation areas related to all the worker IDs stored in the falling risk information database 3.

In a case where the measured value “y” is larger than the average value “Y” (step S105=Yes), the determination unit 23 determines that the falling risk is high (step S106). On the other hand, in a case where the measured value “y” is equal to or smaller than the average value “Y” (step S105=No), the determination unit 23 determines that the falling risk is low (step S107).

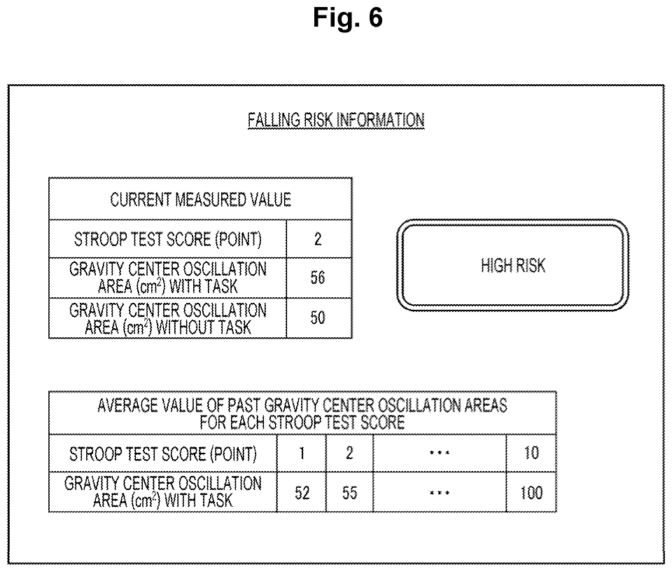

Then, the generation unit 24 generates falling risk information (step S108). The falling risk information includes, for example, a current measured value, a determination result of the falling risk, and an average value of past gravity center oscillation areas. FIG. 6 is a diagram illustrating an example of the falling risk information.

As Illustrated in FIG. 6, the current measured value includes items of the Stroop test score, the gravity center oscillation area with a task (cm²), and the gravity center oscillation area without a task (cm²). The determination result of the falling risk is “high risk”. The average value of past gravity center oscillation areas for each Stroop test score includes items of the Stroop test score and the gravity center oscillation area (cm²) with a task. Since the current measured value 56 is larger than the average value 55, it is determined as “high risk”.

Then, the output unit 15 outputs a report regarding the falling risk information (step S109). The report is transmitted to the falling risk information database 3 via the communication interface 13 and the network 4. The falling risk information database 3 manages and stores falling risk information for each worker ID. The falling risk information database 3 updates the average value of past gravity center oscillation areas using the current measured value. The report stored in the falling risk information database 3 is provided to the worker by an arbitrary method.

Moreover, the output unit 15 causes the display unit 16 to display a report regarding the falling risk information. The worker can check the report displayed on the display unit 16. By viewing the report regarding the falling risk information, the worker can objectively grasp the instability that cannot be recognized by his/her own sense. Moreover, by viewing the report, another worker or a manager can grasp a sign such as wobbling more than usual, and can make risk prediction to grasp a dangerous sign in advance.

### [1-3] Effects of First Embodiment

In the first embodiment, a sensor is attached to a leg portion of a high-place work tool such as a stepladder, and instability of standing position holding in a narrow place such as a stepladder is measured using an evaluation value such as a gravity center oscillation area. Moreover, a Stroop test is carried out on the worker in parallel with the operation of calculating the gravity center oscillation area, and the fatigue level of the worker is measured using the result of the Stroop test. Then, the falling risk is determined by comparing the current measured value with past average value.

Therefore, according to the first embodiment, the falling risk can be determined in consideration of the fatigue level of the worker in a case where the worker works on a stepladder. Moreover, it is possible to determine whether work performance is deteriorated or not in a case where the worker is in high-place work.

Moreover, a report including falling risk information is outputted. As a result, a dangerous sign can be visualized, and the sign can be notified to the worker himself/herself or the surroundings. As a result, the state of the worker can be easily detected while ensuring the safety of the worker.

### [2] Second Embodiment

The second embodiment is another example of a condition for determining the falling risk.

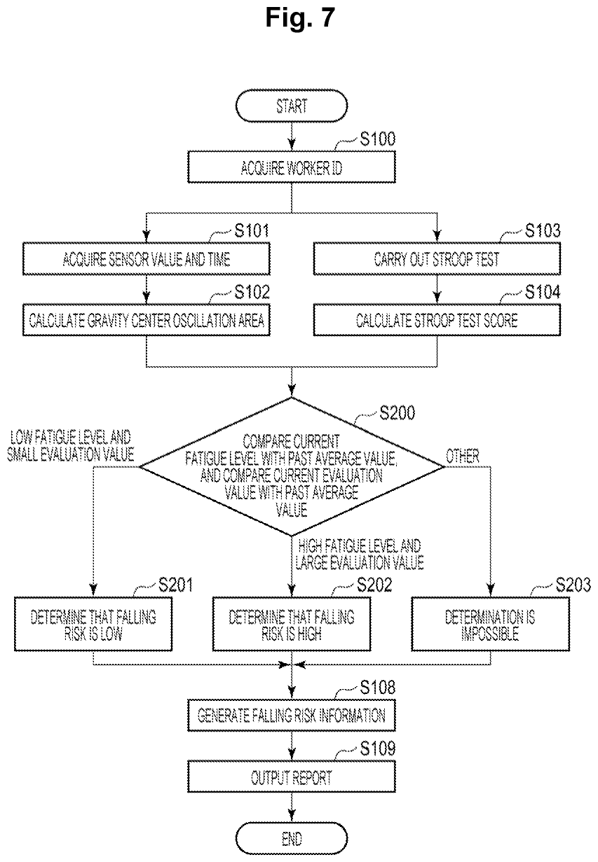

FIG. 7 is a flowchart for explaining an operation of the falling risk determination device 2 according to the second embodiment of the present invention. The operation from steps S100 to S104 is the same as that in the first embodiment.

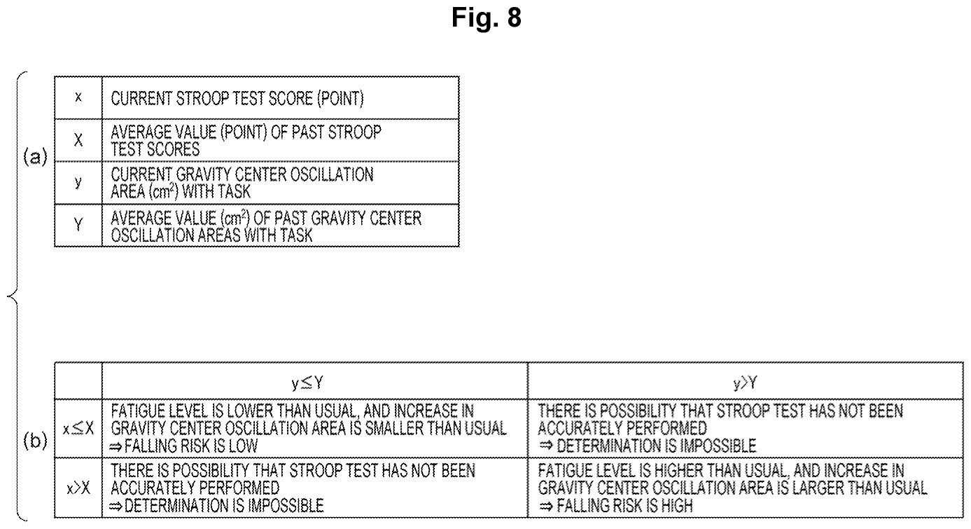

Then, the determination unit 23 determines the falling risk. FIG. 8 is a diagram for explaining a falling risk determination operation by the determination unit 23. FIG. 8(a) is a diagram for explaining the contents of the measured value and the average value used in the determination operation, and FIG. 8(b) is a diagram for explaining a condition for the determination operation.

The current Stroop test score (point) is denoted by “x”, the average value (point) of past Stroop test scores is denoted by “X”, the current gravity center oscillation area (cm²) with a task is denoted by “y”, and the average value (cm²) of past gravity center oscillation areas with a task is denoted by “Y”.

The determination unit 23 compares the current fatigue level with the average value of past fatigue levels, and compares the current evaluation value with the average value of past evaluation values (step S200). In step S200, the current fatigue level is the current Stroop test score “x”. The average value of past fatigue levels is the average value “X” of past Stroop test scores. The current evaluation value is the current gravity center oscillation area “y” with a task. The average value of past evaluation values is the average value “Y” of past gravity center oscillation areas with a task. The average value “X” of past Stroop test scores and the average value “Y” of past gravity center oscillation areas with a task are stored in the memory 11.

Note that the falling risk information database 3 stores the average value “X” of past Stroop test scores and the average value “Y” of past gravity center oscillation areas with a task for each worker ID. In a case where the worker ID is recognized in step S100, the determination unit 23 stores the average value “X” of past Stroop test scores related to the worker ID, and the average value “Y” of past gravity center oscillation areas with a task from the falling risk information database 3 to the memory 11. Moreover, the memory 11 may store the same data as the average value “X” of past Stroop test scores related to all the worker IDs stored in the falling risk information database 3, and the average value “Y” of past gravity center oscillation areas with a task.

In a case where “x≤X and y≤Y” are satisfied in step S200, the determination unit 23 determines that the fatigue level is lower than usual, and the gravity center oscillation area is smaller than usual. In this case, the determination unit 23 determines that the falling risk is low (step S201).

In a case where “x>X and y>Y” are satisfied in step S200, the determination unit 23 determines that the fatigue level is higher than usual, and the gravity center oscillation area is larger than usual. In this case, the determination unit 23 determines that the falling risk is high (step S202).

In a case where “x>X and y≤Y” are satisfied in step S200, the determination unit 23 determines that there is a possibility that the Stroop test has not been accurately performed, and it is considered as an example that the worker has carelessly performed the Stroop test. In this case, the determination unit 23 cannot make a determination. (Step S203).

In a case where “x≤X and y>Y” are satisfied in step S200, the determination unit 23 determines that there is a possibility that the Stroop test has not been accurately performed, and it is considered as an example that the worker concentrates too much on the Stroop test. In this case, the determination unit 23 cannot make a determination. (Step S203).

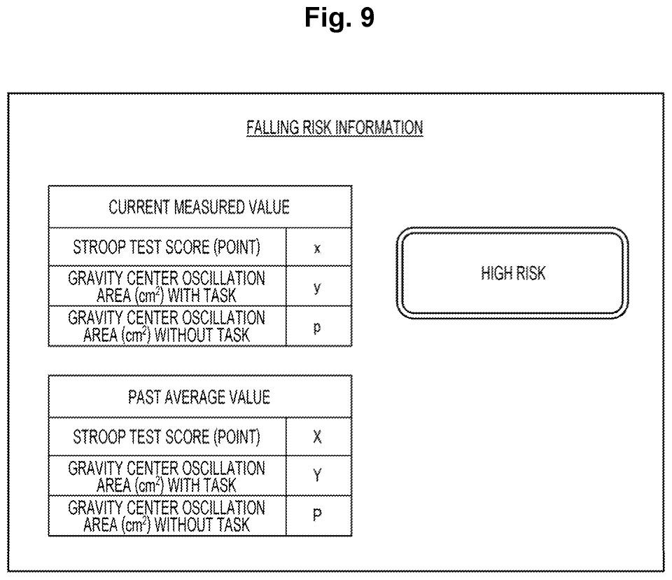

Then, the generation unit 24 generates falling risk information (step S108). The falling risk information includes, for example, a current measured value, a determination result of the falling risk, and a past average value. FIG. 9 is a diagram illustrating an example of the falling risk information.

The current measured value includes items of the Stroop test score “x”, the gravity center oscillation area (cm²) “y” with a task, and the gravity center oscillation area (cm²) “p” without a task. The determination result of the falling risk is “high risk”. The past average value includes items of the Stroop test score “X”, the gravity center oscillation area (cm²) “Y” with a task, and the gravity center oscillation area (cm²) “P” without a task.

Then, the output unit 15 outputs a report regarding the falling risk information (step S109). The report is transmitted to the falling risk information database 3 via the communication interface 13 and the network 4. The falling risk information database 3 manages and stores falling risk information for each worker ID. The falling risk information database 3 updates the average value of past Stroop test scores and the average value of past gravity center oscillation areas. Moreover, the output unit 15 causes the display unit 16 to display a report regarding the falling risk information.

According to the second embodiment, a change in the Stroop test score can be included in a condition for determining the falling risk. Other effects are the same as those of the first embodiment.

### [3] Third Embodiment

The third embodiment is still another example of a condition for determining the falling risk.

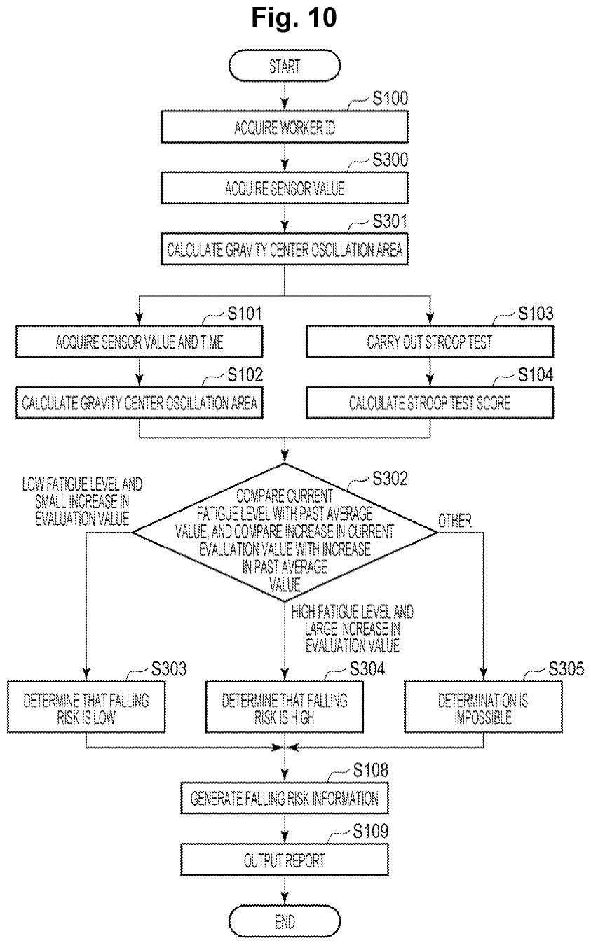

FIG. 10 is a flowchart for explaining an operation of the falling risk determination device 2 according to the third embodiment of the present invention. The operation of step S100 is the same as that of the first embodiment.

Then, the acquisition unit 20 acquires a sensor value related to the weight of the worker from the sensor unit 12 (step S300). Specifically, the acquisition unit 20 continuously acquires the sensor value at regular intervals to acquire time-series data of the sensor value. The sensor value changes according to movement of the gravity center of the worker.

Then, the calculation unit 22 calculates the gravity center of the worker from the sensor value, and calculates an evaluation value (gravity center oscillation area) related to the gravity center oscillation (step S301). In steps S300 and S301, the gravity center oscillation area without a task (without Stroop test) can be calculated.

Then, in steps S101 to S104, the Stroop test is carried out, and the gravity center oscillation area without a task (with Stroop test) is calculated. The operation in steps S101 to S104 are the same as that in the first embodiment.

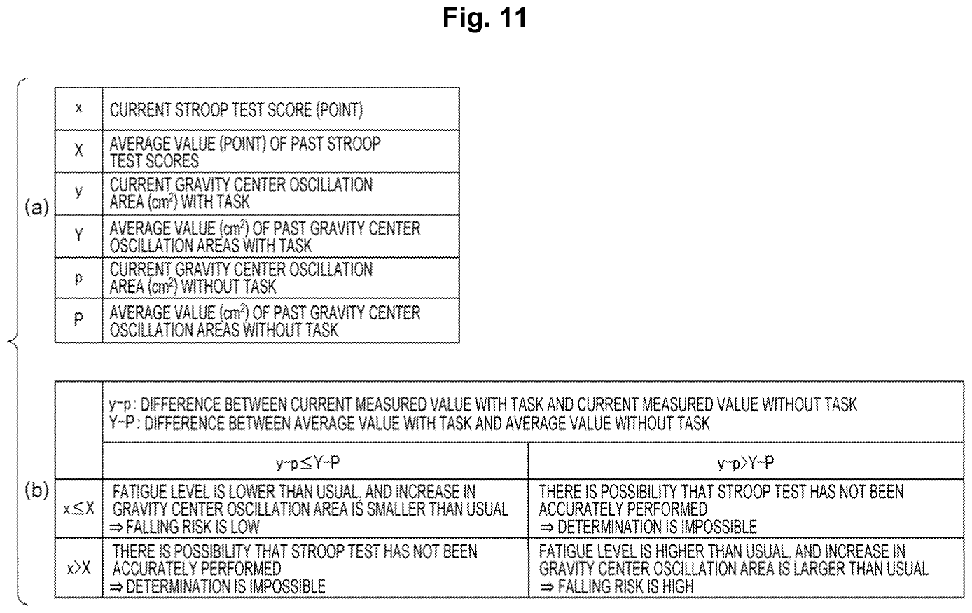

Then, the determination unit 23 determines the falling risk. FIG. 11 is a diagram for explaining a falling risk determination operation by the determination unit 23. FIG. 11(a) is a diagram for explaining the contents of the measured value and the average value used in the determination operation, and FIG. 11(b) is a diagram for explaining a condition for the determination operation.

The current Stroop test score (point) is denoted by “x”, the average value (point) of past Stroop test scores is denoted by “X”, the current gravity center oscillation area (cm²) with a task is denoted by “y”, the average value (cm²) of past gravity center oscillation areas with a task is denoted by “Y”, the current gravity center oscillation area (cm²) without a task is denoted by “p”, and the average value (cm²) of past gravity center oscillation areas without a task is denoted by “P”.

The determination unit 23 compares the current fatigue level with the average value of past fatigue levels, and compares the increase in the current evaluation value with the increase in the average value of past evaluation values (step S302). In step S302, the current fatigue level is the current Stroop test score “x”. The average value of past fatigue levels is the average value “X” of past Stroop test scores. The increase in the current evaluation value is a difference between the current gravity center oscillation area “y” with a task and the current gravity center oscillation area “p” without a task, that is, “y−p”. The increase in the average value of past evaluation values is a difference between the average value “Y” of past gravity center oscillation areas with a task and the average value “P” of past gravity center oscillation areas without a task, that is, “Y−P”. The average value “X” of past Stroop test scores, the average value “Y” of past gravity center oscillation areas with a task, and the average value “P” of past gravity center oscillation areas without a task are stored in the memory 11.

Note that the falling risk information database 3 stores the average value “X” of past Stroop test scores, the average value “Y” of past gravity center oscillation areas with a task, and the average value “P” of past gravity center oscillation areas without a task for each worker ID. In a case where the worker ID is recognized in step S100, the determination unit 23 stores the average value “X” of past Stroop test scores related to the worker ID, the average value “Y” of past gravity center oscillation areas with a task, and the average value “P” of past gravity center oscillation areas without a task from the falling risk information database 3 to the memory 11. Moreover, the memory 11 may store the same data as the average value “X” of past Stroop test scores related to all the worker IDs stored in the falling risk information database 3, the average value “Y” of past gravity center oscillation areas with a task, and the average value “P” of past gravity center oscillation areas without a task.

In a case where “x≤X and y−p≤Y−P” are satisfied in step S302, the determination unit 23 determines that the fatigue level is lower than usual, and the increase in the gravity center oscillation area is smaller than usual. In this case, the determination unit 23 determines that the falling risk is low (step S303).

In a case where “x>X and y−p>Y−P” are satisfied in step S302, the determination unit 23 determines that the fatigue level is higher than usual, and the increase in the gravity center oscillation area is larger than usual. In this case, the determination unit 23 determines that the falling risk is high (step S304).

In a case where the determination unit 23 determines that “x>X and y−p≤Y−P” are satisfied in step S302, there is a possibility that the Stroop test has not been accurately performed, and it is considered as an example that the worker has carelessly performed the Stroop test. In this case, the determination unit 23 cannot make a determination. (Step S305).

In a case where the determination unit 23 determines that “x≤X and y−p>Y−P” are satisfied in step S302, there is a possibility that the Stroop test has not been accurately performed, and it is considered as an example that the worker concentrates too much on the Stroop test. In this case, the determination unit 23 cannot make a determination. (Step S305).

The subsequent operation in steps S108 and S109 is the same as that in the second embodiment.

According to the third embodiment, a change (difference) between the gravity center oscillation area with a task and that without a task can be included in a condition for determining the falling risk. Other effects are the same as those of the first embodiment.

Note that the three types of determination methods described in the first to third embodiments may be selected by a worker operating the input unit 14.

Each processing according to the above-described embodiments can also be stored as a program that can be executed by a processor that is a computer. In addition, the program can be distributed by being stored in a storage medium of an external storage device such as a magnetic disk, an optical disk, or a semiconductor memory. Then, the processor reads the program stored in the storage medium of the external storage device, and the operation is controlled by the read program, whereby the above-described processing can be executed. Moreover, the program can also be provided through a network.

The present invention is not limited to the above embodiments, and various modifications can be made in the implementation stage without departing from the gist thereof. Moreover, the embodiments may be implemented in appropriate combination, and in that case, a combined effect can be obtained. Furthermore, the above-described embodiments include various inventions, and various inventions can be extracted by a combination selected from a plurality of disclosed components. For example, even if some components are deleted from all the components shown in an embodiment, a configuration from which the components are deleted can be extracted as an invention in a case where a problem can be solved and an effect can be obtained.

### REFERENCE SIGNS LIST

1 Falling risk determination system
2 Falling risk determination device
3 Falling risk information database
4 Network
10 Processing circuit
11 Memory
12 Sensor unit
13 Communication interface
14 Input unit
15 Output unit
16 Display unit
17 Bus
20 Acquisition unit
21 Fatigue level measurement unit
22 Calculation unit
23 Determination unit
24 Generation unit
30 Stepladder
31 Leg
32 Sensor

1. A falling risk determination device comprising:
a processor; and
a storage medium having computer program instructions stored thereon, when executed by the processor, perform to:
acquire time-series data related to gravity center oscillation of a worker from a sensor unit provided at a leg portion of a high-place work tool on which the worker rides;
calculate an evaluation value related to the gravity center oscillation from the time-series data;
carry out a Stroop test on the worker in parallel with processing of acquiring the time-series data, and measure a fatigue level of the worker using a result of the Stroop test; and
determine that a falling risk is high in a case where the evaluation value is larger than an average value of past evaluation values corresponding to the fatigue level.

2. The falling risk determination device according to claim 1,
wherein the computer program instructions further perform to calculates a gravity center oscillation area of the worker as the evaluation value.

3. The falling risk determination device according to claim 1,
wherein the computer program instructions further perform to unit measures a Stroop test score as the fatigue level.

4. The falling risk determination device according to claim 1,
wherein the computer program instructions further perform to determines that a falling risk is high in a case where the fatigue level is higher than an average value of past fatigue levels.

5. The falling risk determination device according to claim 1, wherein the computer program instructions further perform to generate falling risk information including the fatigue level, the evaluation value, the average value, and a falling risk determination result.

6. A falling risk determination method comprising the steps of:
acquiring time-series data related to gravity center oscillation of a worker from a sensor unit provided at a leg portion of a high-place work tool on which the worker rides;
calculating an evaluation value related to the gravity center oscillation from the time-series data;
carrying out a Stroop test on the worker in parallel with processing of acquiring the time-series data and measuring a fatigue level of the worker using a result of the Stroop test; and
determining that a falling risk is high in a case where the evaluation value is larger than an average value of past evaluation values corresponding to the fatigue level.

7. A non-transitory computer-readable medium having computer-executable instructions that, upon execution of the instructions by a processor of a computer, cause the computer to function as the falling risk determination device according to claim 1.
